(19) United States
(12) Patent Application Publication
(10) Pub. No.: US 2011/0020291 A1
(43) Pub. Date: Jan. 27, 2011

(54) USE OF STEM CELLS FOR WOUND HEALING

(76) Inventors: Debrabrata Banerjee, Bellerose, NY (US); Prasun J. Mishra, Gaithersburg, MD (US); Pravin J. Mishra, Edsion, NJ (US); Joseph R. Bertino, Branford, CT (US); Rita Humeniuk, Gaithersburg, MD (US); John Glod, North Brunswick, NJ (US)

Correspondence Address: FOX ROTHSCHILD LLP;PRINCETON PIKE CORPORATE CENTER, 997 LENOX DRIVE, BLDG. #3, LAWRENCEVILLE, NJ, 08648, US

(21) Appl. No.: 12/743,309

(22) PCT Filed: Nov. 15, 2008
(86) PCT No.: PCT/US08/83711

§ 371 Date: Sep. 17, 2010

Related U.S. Application Data
(60) Provisional application No. 61/003,343, filed on Nov. 17, 2007.

(51) Int. Cl. A61K 35/12; C12N 5/0775; A61P 17/02

(52) U.S. Cl. 424/93.7; 435/325; 435/366; 435/377; 424/520

(57) ABSTRACT

Cells, compositions, and methods of cell therapy for administering a therapeutically effective amount of stem cells or cell concentrate to achieve accelerated wound healing of normal and chronic wounds, while minimizing the formation of scar tissue.

### CROSS REFERENCE TO RELATED APPLICATIONS

The present application claims priority under 35 U.S.C. 119(e) to U.S. Provisional Patent Application No. 61/003,343, which was filed on Nov. 17, 2007 and is incorporated herein by reference.

### FIELD OF THE INVENTION

The present invention provides cells, compositions, and methods of cell therapy to accelerate wound healing of normal and chronic wounds, while minimizing the formation of scar tissue, by administering to an affected subject a therapeutically effective amount of stem cells or cell concentrate.

### BACKGROUND OF THE INVENTION

Acute and chronic wounds remain difficult to treat, despite a better understanding of the cellular and molecular biology of wound healing and advances in wound dressing and care. Wound healing is a complex but well coordinated process comprising an inflammatory reaction, a proliferative process leading to tissue restoration, angiogenesis and formation of extracellular matrix accompanied by scar tissue remodeling. Cellular participants as well as multiple growth factors and cytokines released by the cells at the wound site regulate these processes and ultimately facilitate wound closure. Deregulated healing process often delays these repair pathways and may eventually lead to chronic wounds, such as in diabetics, that are difficult to heal. Deregulation may also result in excessive fibrosis leading to keloid formation. While there has been an increase in the understanding of underlying biologic principles of chronic wounds and significant scientific developments in the use of recombinant growth factors, use of bioengineered skin equivalents and overall improvement in standards of wound care, treatment of chronic wounds remains difficult. This has stimulated investigation of alternative therapeutic modalities involving somatic stem cells including bone marrow derived human mesenchymal stem cells (BMD-hMSCs).

The bone marrow is known to harbor two major types of stem cells, the hematopoietic stem cell (HSC) and the non-hematopoietic or mesenchymal stem cell (MSC). Under appropriate culture conditions, MSCs can give rise to cells of muscle, bone, fat, and cartilage lineage. Like true stem cells, MSCs have the capacity for self-renewal and differentiation, and based on this potential, MSCs hold promise for clinical applications for regenerative medicine as well as for use as delivery vehicles. Most recently, bone marrow derived MSCs have been shown to differentiate into myofibroblast-like cells that resemble carcinoma associated myofibroblasts when exposed to tumor cell conditioned medium for prolonged periods of time.

Cell differentiation into myofibroblast-like cells is relevant to wound healing because myofibroblasts are specialized fibroblastic cells that appear transiently during skin wound healing but persist in and remain overactive in fibrocontractive diseases such as hypertrophic scars. In vivo, myofibroblasts are responsible for generation of mechanical forces that allow proper granulation tissue contraction and wound healing. Matrix contraction depends both on alpha-smooth muscle actin (α-SMA) expression within cellular stress fibers and assembly of large focal adhesions linking myofibroblasts to the matrix. The contractile forces generated during human dermal wound healing are thought to be due to the differentiation of human dermal fibroblasts (HDFs) into smooth muscle-like cells called human dermal myofibroblasts (HDMs). HDMs are distinguishable from HDFs by their structural features and expression of alpha-smooth muscle actin stress fibers.

Of particular relevance to wound healing, is the fact that MSCs are also known to migrate to various in vivo locations, including sites of hematopoiesis such as the bone marrow, sites of inflammation and sites of injury. The ability of MSCs to migrate to areas of injury suggests that they may play a role in the recovery process following injury. Recent research has shown that there is an increase in the number of circulating mesenchymal bone marrow stem cells in peripheral blood of patients with severe burns as compared with normal donors. Moreover, systemically administered MSCs have been shown to improve recovery in animal models of stroke and myocardial infarction. Such studies, combined with the known uses of myofibroblast cells, encourage investigation of MSC differentiation into myofibroblast-like cells for use in wound healing.

To date, however, there has been no study of and no data available regarding the efficacy of MSCs for use in wound healing. Indeed, there is a need for a more complete understanding of the mechanism of action of MSCs in the wound healing process and how such MSCs can facilitate and/or accelerate the wound healing process. As set forth herein, the present invention addresses such a need and provides supporting data for the efficacy of MSC differentiation and acceleration of the wound healing process, with minimal scar tissue formation.

### SUMMARY OF THE INVENTION

The present invention provides cells, compositions, and methods of cell therapy for administering to an affected subject a therapeutically effective amount of stem cells or cell concentrate to achieve accelerated wound healing of normal and chronic wounds, while minimizing the formation of scar tissue. As provided herein, the stem cells of the present invention differentiate into myofibroblast-like cells upon exposure to one or more signaling molecules of a keratinocyte cell population. Accordingly, in one embodiment, a multipotent stem cell of the present invention (e.g. a mesenchymal stem cell) may be administered directly to the wound site of a patient such that migration and differentiation into myofibroblast-like cells occur in response to signaling molecules presented in vivo. Alternatively, the stem cells of the present invention may be incubated with conditioned medium from a keratinocyte cell population and/or communication molecules from a keratinocyte cell population to induce in vitro differentiation of the stem cells into dermal myofibroblast-like cells. These differentiated cells may then administered to the wound site of the patient to, inter alia, optimize the proliferation of both myofibroblast cells and pancytokeratin positive cells within the wound. In an even further alternative, lysates of either the myofibroblast-like cells of the present invention or MSC cells, including the communication molecules associated therewith, may be directly administered to the wound site of the patient to, inter alia, optimize the proliferation of both myofibroblast cells and pancytokeratin positive cells within the wound. In certain embodiments, these lysates may be co-administered with a multi-potent stem cell of the present invention.

In each of the foregoing embodiments, the compositions and methods discussed herein provide for accelerated wound healing, as determined by quantitative measurements of wound area relative to wound healing without the composition and methods of the present invention. Furthermore, the compositions and methods of the present invention for provide for minimized residual scarring associated with the wound.

The stem cells of the present invention may be utilized to effectively populate the wounded area because of their multipotent or phenotypically broad differentiation potential, particularly the ability to differentiate into myofibroblast-like cells. While not limited thereto, preferred stem cells include mesenchymal stem cells (MSC), which are typically, but not exclusively, derived from human bone marrow aspirate. The stem cells of the present invention may also include any other type of stem cells including, but not limited to HSCs, human embryonic stem cells, murine embryonic stem cells, stem cells isolated from human or murine umbilical cord blood, and the like. Stem cells may be obtained from organisms, blastocysts, or cells isolated or created by suitable means known in the art. In other embodiments, the stem cells are adult multipotent stem cells or other stem cells that are able to give rise to myofibroblast-like cells when administered or cultured according to the methods described herein.

The stem cells may be derived from any source that is compatible with the uses described herein. By way of example only, such a source may include the bone marrow of a human source, such as from an immunocompatible donor or autologously from the patient. While autologous cells are preferred, the present invention is not limited to this source and any stem cell may be used as contemplated herein.

In one embodiment, a therapeutically effective amount of the stem cells (e.g. hMSCs) may be directly administered to the subject such that the cells differentiate into myofibroblast-like cells in vivo. While a therapeutically effective amount may be between 2.5×10⁵ to 1.0×10⁷ per 30-50 mm² of the wound, the present invention is not limited to this amount and may be based on a set amount, the weight of the patient, or any other amount sufficient to accelerate the wound healing process, as described herein.

In another embodiment, the stem cells (e.g. hMSCs) of the present invention may be differentiated into a myofibroblast-like cell in vitro, then administered to the patient. For example, the hMSCs of the present invention may be cultured in the presence of keratinocyte conditioned medium (KCM), or any similar medium having one or more cytokines including interleukin-8 (IL-8), interleukin-6 (IL-6), vascular endothelial growth factor (VEGF), stromal cell-derived factor-1 (SDF-1), chemokine (C—X—C motif) ligand 5 (CXCL5) and combinations thereof.

The myofibroblast-like cells resulting from the foregoing hMSC differentiation express numerous cytokines and cytoskeletal proteins. These cytokines include, but are not limited to, one or more of IL-6, IL-8, SDF-1, CXCL5, VEGF, MMP1, CXCL6, COL4A4, MMP13, CYP7B1, ADAMDEC1, SLC6A1, CXCL1, PF4V1, CXCL3, CH25H, SFRP2, DARC, HCK, ERC2, CLIC6, BCL8 and combinations thereof. The cytoskeletal proteins include, but are not limited to, one or more of vinculin, F-actin filaments, vimentin, fibroblast surface proteins, increased production of α-smooth muscle actin and combinations thereof.

Once differentiated, a therapeutically effective amount of the myofibroblast-like cells may be administered at or near the wound site of the patient. While a therapeutically effective amount may be between 2.5×10⁵ to 1.0×10⁷ per 30-50 mm² of the wound, the present invention is not limited to this amount and any amount may be administered that is sufficient to accelerate the wound healing process, as described herein.

In further embodiments of the present invention, a therapeutically effective amount of a cell lysate of either differentiated myofibroblast-like cells or MSCs may be directly administered at or near the wound site of the patient to accelerate wound healing and minimize scar tissue formation. While not limited thereto, such lysates may include one or more cytokines including, but not limited to, IL-6, IL-8, SDF-1, CXCL5, VEGF, MMP1, CXCL6, COL4A4, MMP13, CYP7B1, ADAMDEC1, SLC6A1, CXCL1, PF4V1, CXCL3, CH25H, SFRP2, DARC, HCK, ERC2, CLIC6, BCL8 and combinations thereof. While a therapeutically effective amount may be lysate obtained from approximately 5.0×10⁶ cells per 30-50 mm² of the wound, the present invention is not limited to this amount and any amount may be administered that is sufficient to accelerate the wound healing process, as described herein. In further embodiments, the lysate amy be co-administered with one or a population of MSCs or myofibroblast-like cells of the present invention.

As provided herein, the stem cells of the present invention may be administered using any method known in the art. For example, each of the foregoing embodiments may be administered by subcutaneous injection, applied topically, implanted within either a preformed or in situ formed matrix, or by any other suitable means known in the art. Additionally, the cells and compositions of the present invention may be administered with one or more biological agents. Such biological agents may include, but are not limited to, antifungal agents, antibacterial agents, anti-viral agents, anti-parasitic agents, growth factors, steroids, pain medications (e.g. aspirin, and NSAID, and/or local anesthetic), anti-inflammatory agents, angiogenic factors, anesthetics, mucopolysaccharides, metals, adjuvants, cells, agents useful in the repair of tissue, bone, and vascular injury, other known wound healing agents, and combinations thereof.

Additional embodiments and features of the present invention will be apparent to one of ordinary skill in the art based upon description provided herein.

### BRIEF DESCRIPTION OF THE FIGURES

FIG. 1 illustrates the area of skin wounds on the back of nude mice, shown (from day 1-5), where the wound+hMSC group was healed without much seen scar within a week compared to wound+saline and only hMSc injected group.

FIG. 2 illustrates the area of skin wounds on the back of diabetic mice (from day 115), where hMSC treated wounds showed rapid wound closure (day 6) and were rapidly healed compared to natural wound healing (wound closure was seen on day 15) in the diabetic mice.

FIG. 3 illustrates the measurement of wound healing using area of ellipse formula (0.5×length of Major axis) (0.5×length of Major axis) (π) WYSOCKI A: Wound measurement. Int J Dermatol 35: 82-91, 1996.

FIG. 4 illustrates MSC migration to the injury site and dermal myofibroblast like differentiation after exposure to KCM. FIG. 4(A) provides hMSCs labeled (CFDASE) and injected at the periphery of wounded skin subcutaneously after 48 hour hMSCs (green) were found to migrate to the injury site. FIG. 4(B) illustrates that hMSCs were found to migrate toward keratinocytes as well as to KCM in greater numbers than to control medium using transwell chamber migration assay. FIG. 4(C) illustrates a merge confocal image of KCMSCs stained for vinculin (green) and phalloidin (red). The focal adhesions (green) appear to hold down actin stress fibers (red). Inset shows actin filaments terminating with vinculin at the cell periphery FIG. 4(D) illustrates KGMSCs showing diffused vinculin staining when compared with KCMSCs. FIG. 4(E) illustrates naïve hMSCs stained for vinculin and phalloidin as a control. FIGS. 4(F)-4(G) illustrate differentiated KCMSCs and KGMSCs stained for α-SMA (FIG. 4(F)); a FSP (FIG. 4(G)) and Vimentin (FIG. 4(H)). FIG. 4(I) provides a Graph showing Quantitative analysis of KCMSC and KGMSC expressing markers α-SMA, FSP and vimentin.

FIG. 5 illustrates contraction of collagen gel by hams and KCMSC using Floating collagen gel contraction (FCGC) assay. Increased fold change of SDF-1 mRNA expression levels in KCMSC and KGMSC by q-RTPCR and cytokine profiling of KCMSCs and KGMSCs FIG. 5(A) provides a FCGC assay was performed, 6×10⁴ cells (hMSCs, KCMSCs and KGMSCs) were mixed with collagen gel was contracted significantly after 48 h compared with no treatment; FIG. 5(B) provides a schematic representation of the FCGC. FIG. 5(C) illustrates a bar graph (measured and plotted using ImageJ; publicly available NIH Image program) depicting the contraction comparison between KCMSC, KGMSC, nïve hMSC and no treatment. FIG. 5(D) illustrates increased mRNA expression levels of SDF-1 in KCMSC and KGMSC were determined using q-RTPCR. FIG. 5(E) illustrates Cytokine profiling of conditioned medium from keratinocyte, hMSC, KCMSC and KGMSC was performed using Multiplex assay and secreted levels were observed for IL-6, IL-8, SDF-1 and VEGF.

FIG. 6 illustrates a comparative gene expression analysis of KCMSc and KGMSCs. FIG. 6(A) provides a heat map showing top 20 upregulated genes in KCM treated MSCs versus KGM treated MSCs. The expression levels of individual transcripts are shown from green (low) to red (high). FIG. 6(B) provides a pie chart showing the KEGG pathways containing a significant percentage of the top 300 genes up-regulated in KCMSC vs KGMSC. The pathways were assigned a statistical score based on the Fisher test and sorted clockwise from the inflammation mediated by chemokine and cytokine. The area of an individual slice represents the percentage of the top 300 genes up-regulated in KCMSC.

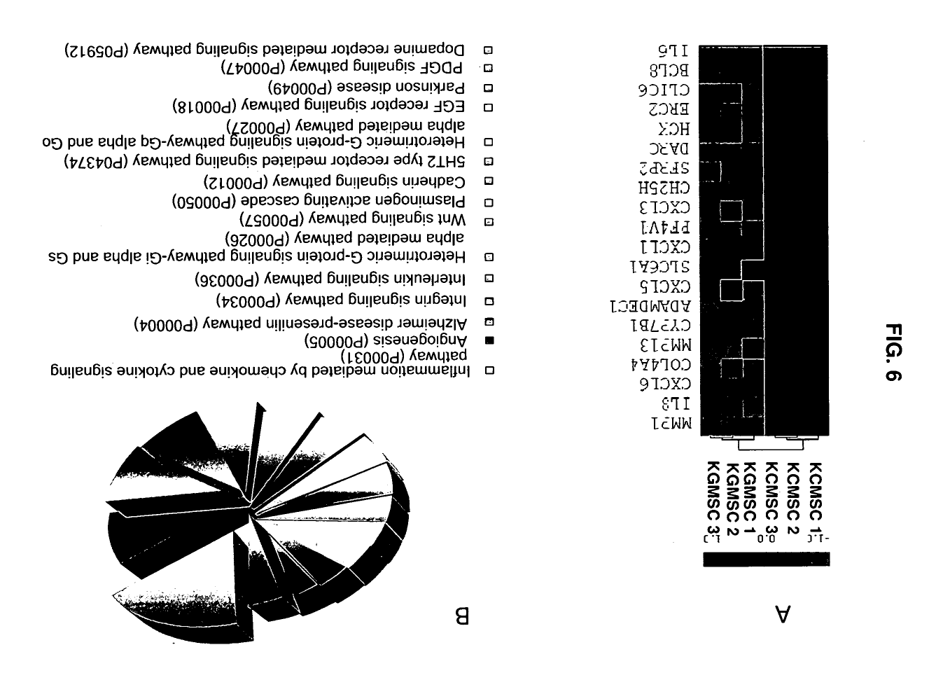

FIG. 7 illustrates (1) H&E and Immunohistochemical (Cytokeratin 17 and Pancytokeratin) staining of skin sections shows restoration of both dermis and epidermis in skins of mice treated with hMSC, hMSC lysate and KCMSC as compared with controls; (2) RT-PCR analysis of KCMSC and KGMSC; (3) increased fold change of SDF-1 and CXCL5 mRNA expression levels in KCMSC also increased level in wounded skin RNA injected with hMSC and hMSC lysate. FIGS. 7(A-C) show the normal (unwounded) skin and FIGS. 7(D-F) show wounded skin sections at day 1. FIGS. 7(G-I) show that the wounded skin was allowed to heal naturally (after 8 days). FIGS. 7(J-L) illustrate large number of pancytokeratin positive cells were observed in the dermis of hMSC administrated wounded skin; and FIGS. 7(M-O) illustrate KCMSC injected skin section showing positive staining for cytokeratin 17 and pancytokeratin. FIGS. 7(P-R) illustrate hMSC Lysate injected skin sections compared with FIGS. 7(S-U), WI38 injected wounded skin sections. FIG. 7(V) illustrates that the PCR product was analysed on agarose gel for SDF-1, CXCL5, vimentin, VEGF and GAPDH was used as an internal control. Significant Increase observed in expression levels of CXCL5 and SDF-1 in KCMSCs compared with KGMSCs. FIG. 7(W) illustrates in hMSC and hMSC lysate injected skin (wounded) mRNA expression levels of SDF-1 and CXCL5 was increased compared with naturally healing (wounded) skin and normal skin GAPDH was utilized as an internal control.

FIG. 8 illustrates accelerated wound healing by hMSC, hMSC lysate and KCMSC in nu/nude mice and NOD-SCID mice with FIG. 8(A) providing a macroscopic observation of hMSC and hMSC lysate injected wounds at different time intervals in nude mice compared with naturally healing group with FIG. 5(1A) illustrating a bar graph representation of wound closure after 1, 3, 6, 8, 10 and 13 days in nude mice. FIG. 8(B) shows NOD-SCID mice were injected with hMSC and hMSC lysate, observed for wound closure at different time point which was compared with naturally healing group with FIG. (8B) illustrating a Graphical representation of wound closure after 1, 3, 6, 8, 10 and 13 days. FIG. 8(C) provides a comparative wound closure observation of hMSC, KCMSC, WI38 injected and naturally healing nude mice with FIG. 8(1C) illustrating after log time observation (40 days) less or no residual scarring was seen in hMSC injected mice whereas KCMSC injected mice also demonstrated less residual scarring compared with WI38 injected mice and naturally healing mice and FIG. 8(2C) depicting a bar graph depict comparative wound closure at different time intervals.

FIG. 9 illustrates conditioned medium concentrate (CMC) from hMSC, KCMSC and KGMSC also contribute significantly in wound healing along with naive hMSCs which can also accelerate wound closure in deep wounds. FIG. 9(A) provides comparative wound closure in KCMSC(CMC), KGMSC(CMC) and MSC(CMC) Injected wound with FIG. 9(1A) illustrating less or no residual scarring was observed in MSC(CMC), KCMSC(CMC) when compared to, KGMSC(CMC) and naturally healing mice and FIG. 9(2A) illustrating that the wound area was measured and plotted at different time intervals. FIG. 9(B) provides that a deep wound was made aseptically and hMSC was injected at the periphery and observed on different scheduled time point with FIG. 9(1B) illustrating long term follow up revealed less or no residual scarring in hMSC injected deep wound compared with naturally healing wound and FIG. 9(2B) providing a bar graph representation of wound closure at different time intervals. FIG. 9(C) illustrates a schematic representation of deep wound, axes of sections and the area of interest.

### DETAILED DESCRIPTION OF THE INVENTION

The present invention provides cells, compositions, and methods of cell therapy comprising administering to an affected subject a therapeutically effective amount of stem cells or cell concentrate to achieve accelerated wound healing of normal and chronic wounds, while minimizing the formation of scar tissue. As provided herein, the stem cells of the present invention differentiate into myofibroblast-like cells upon exposure to one or more signaling molecules of a keratinocyte cell population. Accordingly, in one embodiment, a multipotent stem cell of the present invention (e.g. a mesenchymal stem cell) may be administered directly to the wound site of the patient such that migration and differentiation into myofibroblast-like cells occur in response to signaling molecule present in vivo. Alternatively, the stem cells of the present invention may be incubated with conditioned medium from a keratinocyte population, including one or more associated communication molecules, to induce in vitro differentiation of the stem cells into dermal myofibroblast-like cells. These differentiated cells may then administered to the wound site of the patient to optimize the proliferation of both myofibroblast cells and pancytokeratin positive cells within the wound. In an even further alternative, lysates of the either myofibroblast-like cells or MSCs of the present invention, including the cytokines associated therewith, may be administered to the wound site of the patient to optimize the proliferation of both myofibroblast cells and pancytokeratin positive cells within the wound.

In each of the foregoing embodiments, the cells and methods discussed herein provide for accelerated wound healing, as determined by quantitative measurements of wound area relative to natural wound healing without the addition of the cells and compositions of the present invention. Furthermore, the cells and methods of the present invention for provide for minimized residual scarring associated with the wound.

As used herein, the terms “stem cell” and “mesenchymal stem cell” relate to cells having developmental plasticity that are able to produce other cell types than the cells from which the stem cells are derived. To this end, they refer to multipotent cells able differentiate into a variety of cell types.

As also used herein, the term “myofibroblast-like cells” relates to cells characterized by expression of one or more cytoskeletal markers including vinculin, F-actin filaments, vimentin, fibroblast surface proteins, as well as increased production of α-smooth muscle actin. These cells may be further characterized by expression and secretion of one or more cytokines including IL-6, IL-8, VEGF, CXCL5, SDF-1, MMP1, CXCL6, COL4A4, MMP13, CYP7B1, ADAMDEC1, SLC6A1, CXCL1, PF4V1, CXCL3, CH25H, SFRP2, DARC, HCK, ERC2, CLIC6, BCL8 and combinations thereof.

As also used herein, the term “wound” relates to damage, tearning, cutting, or puncturing of the epithelial tissue of the body, particularly the skin, wherein the wound is caused by an event such as disease, trauma, surgery, burns, bites or the like. Such wounds may include, but are not limited to, abrasions, avulsions, blowing wounds, burn wounds, contusions, gunshot wounds, incised wounds, open wounds, penetrating wounds, perforating wounds, puncture wounds, seton wounds, stab wounds, surgical wounds, subcutaneous wounds, diabetic lesions, tangential wounds, or the like.

The stem cells of the present invention may be utilized to effectively populate the wounded area because of their multipotent or phenotypically broad differentiation potential, particularly the ability to differentiate into myofibroblast-like cells. While not limited thereto, preferred stem cells include mesenchymal stem cells (MSC), which are, most preferably, derived from human bone marrow aspirate.

The MSCs of the present invention may be isolated using any method known in the art. By way of example only, in one embodiment, the MSCs may be isolated from a bone marrow aspirate using a gradient to eliminate unwanted cell types. In one embodiment, the MSC may be isolated by adhering to a culture dish, while essentially all other cell types remain in suspension or are removed from the MSCs, as taught within Friedenstein, Exp. Hematol. 4:267-74, 1976 the contents of which are incorporated herein by reference. After discarding the non-adherent cells, MSC are grown and expanded in culture, yielding a well defined population of pluripotent stem cells. Culture media may be comprised of Mesencult media with MSC stimulatory supplements and Fetal Bovine Serum (FBS), or any other type of culture media known in the art for establishing an MSC cell line. Established cultures may then be grown in minimum essential medium (MEM) preferably containing 10% FBS and an antimicrobial agent (e.g. penicillin and/or streptomycin). Each resulting cell line may be tested for myogenic, osteogenic and adipogenic differentiation to confirm multipotency, and subcultured and/or frozen in liquid nitrogen until use. One of ordinary skill in the art will appreciate, however, that additional or alternative methods of stem cell isolation are available and that such methods may be substituted to the foregoing to achieve the same result.

The MSCs may be derived from any source that is compatible with the uses described herein. By way of example only, such a source may include a human source, such as from an immunocompatible donor or autologously from the patient. While autologous human MSCs (hMSC) are preferred, as these cell types eliminate major immunotolerance concerns, the present invention is not limited to this source and any source of MSCs may be used as contemplated herein.

In alternative embodiments of the present invention, the pluripotent stem cell population is comprised, instead, of hematopoietic stem cells (HSCs), which may be derived from the bone marrow, peripheral blood, or other known sources. The HSCs are isolated from a healthy and compatible donor, preferably autologously, using techniques commonly known in the art.

The present invention is not limited to the foregoing MSC and HSC stem cells. Rather, any type of stem cell or multipotent cell may be used in accordance with the present invention. Such stems cells may include any multipotent, pluripotent, or totipotent stem cells known in the art. For example, the stem cells may be human embryonic stem cells, murine embryonic stem cells, or other mammalian stem cells. Alternatively, stem cells may be isolated from human or murine umbilical cord blood or anyone other means associated with obtaining such cells. To this end, cells may be obtained from organisms, blastocysts, or cells isolated or created by suitable means known in the art. In other embodiments, the stem cells are multipotent adult stem cells and other stem cells that are able to give rise to myofibroblast-like cells when administered or cultured according to the methods described herein. In accordance with the foregoing, while the stem cells herein are referred to as “MSCs” or “hMSCs,” one of ordinary skill in the art will appreciate that these stem cells may be interchanged with any of the foregoing alternative types in accordance with the present invention.

Regardless of origin, and as noted above, in one embodiment, a therapeutically effective amount of stem cells (e.g. MSCs) may be isolated and directly administered to the subject such that the cells differentiate into myofibroblast-like cells in vivo. In one embodiment, between 2.5×10⁵ to 1.0×10⁷ MSCs per approximately 30-50 mm² of the wound may be administered subcutaneously at or near the wound area of the patient. In a further embodiment, between 2.5×10⁵ to 1.0×10⁶ MSCs may be administered per approximately 30-50 mm² of the wound area. In even further embodiments, approximately 5.0×10⁵ MSCs per 30-50 mm² of the wound may be administered subcutaneously at or near the wound area of the patient. The therapeutically effective amount of MSCs, however, is not necessarily limited to the foregoing ranges or numbers of cells. For example, the number of cells administered may be a function of the body weight of the patient, with effective amount ranging from, but not limited to, 1×10⁷ to 1×10⁸ cells per kg of body weight. In even further embodiments, a therapeutically effective amount, as used herein, refers to an amount sufficient to accelerate the wound healing process, as described herein. To this end, any number of cells may be administered such that they achieve the effects contemplated herein.

In another embodiment, the MSCs of the present invention may be isolated and differentiated into a myofibroblast-like cell in vitro, then administered to the patient. For example, in one embodiment the MSCs of the present invention may be cultured in the presence of keratinocyte conditioned medium (KCM) and/or one or more communication molecules (i.e. cytokines) associated therewith. As used herein, “keratinocyte conditioned medium” or “KCM” includes the conditioned medium harvested from cultures epithelial cells by any means known in the art. In one embodiment, for example, KCM may be derived from a primary keratinocyte cell line of epithelial cells, preferably human epithelial cells. These cells are cultured in keratinocyte growth medium (KGM), (e.g. C-20011 obtained from Promo cell GmbH, Germany) or any other cell growth medium known in the art for culturing keratinocyte cell populations. The keratinocyte cells are incubated on KGM and the resulting KCM may be harvested, centrifuged, and filtered. Such conditioned medium includes, but is not limited to, the cytokines and other communication molecules associated with keratinocyte proliferation.

In one embodiment, cytokines associated with KCM include, but are not limited to, interleukin-8 (IL-8), interleukin-6 (IL-6), vascular endothelial growth factor (VEGF), stromal cell-derived factor-1 (SDF-1), chemokine (C-X-C motif) ligand 5 (CXCL5), or combinations thereof. Preferably, cytokines associated with KCM that induce myofibroblast differentiation include, but are not limited to, IL-6 and IL-8. To this end, as used herein, “KCM” and “conditioned medium” may also be defined as any medium having any one or more of the foregoing cytokine molecules that may be used to differentiate nïve MSCs into myofibroblast-like cells.

The MSCs of the present invention may be exposed to or incubated with the KCM, in vitro, to induce myofibroblast-like differentiation. The MSCs may be incubated for any length of time to induce differentiation. In a non-limiting example, adequate differentiation of the MSCs is detected when the MSCs are incubated between 10 and 30 days, most preferably for approximately 30 days. At the end of the incubation period, the resulting myofibroblast-like cells are collected and concentrated.

The resulting myofibroblast-like cells exhibit various cytokines and cytoskeletal proteins associated with myofibro-blast-like cells. The cytokines include, but not limited to, one or more of IL-6, IL-8, SDF-1, CXCL5, VEGF, MMP1, CXCL6, COL4A4, MMP13, CYP7B1, ADAMDEC1, SLC6A1, CXCL1, PF4V1, CXCL3, CH25H, SFRP2, DARC, HCK, ERC2, CLIC6, BCL8 or combinations thereof. Each of these cytokines may be expressed within and secreted from the myofibroblast-like cells within the range of approximately 0-2,300.00 pg/ml, with a more preferred range being between 225.00-2,300.00 pg/ml. In one embodiment, IL-6 is expressed between 800.00-900.00 pg/ml and IL-8 is expressed between 450.00-2,300.00 pg/ml. In another embodiment, VEGF is expressed between 1,600.00-2,300.00 pg/ml and SDF-1 is expressed between 225.00-1,300.00 pg/ml. While not intending to be bound by theory, these cytokines, particularly IL-6 and IL-8 are thought to control MSC recruitment and differentiation into myofibroblasts, while CXCL5 is thought to control the proliferation of pancytokeratin positive cells.

The cytoskeletal proteins include, but are not limited to, one or more of vinculin, F-actin filaments, vimentin, fibroblast surface proteins, as well as increased production of α-smooth muscle actin. In one embodiment, greater than 29% of the differentiated hMSCs express α-smooth muscle actin. In another embodiment, approximately 75% of the differentiated MSCs expressed α-smooth muscle actin.

Once the MSCs are differentiated, in accordance with the foregoing, a therapeutically effective amount of the myofibroblast-like cells may be administered at or near the wound site of the patient. In one embodiment, between 2.5×10⁵ to 1.0×10⁷ of the myofibroblast-like cells per 30-50 mm² of the wound are administered subcutaneously at or near the wound of the patient. In a further embodiment, between 2.5×10⁵ to 1.0×10⁶ of the myofibroblast-like cells may be administered per approximately 30-50 mm² of the wound area. In even further embodiments, approximately 5.0×10⁵ of the myofibroblast-like cells per 30-50 mm² of the wound may be administered subcutaneously at or near the wound area of the patient. The therapeutically effective amount of the myofibroblast-like cells, however, is not necessarily limited to the foregoing ranges or numbers of cells. For example, the numbers of cells administered may be a function of the body weight of the patient, with effective amount ranging from, but not limited to, 1×10⁷ to 1×10⁸ cells per kg of body weight. The therapeutically effective amount of differentiated MSCs or myofibroblast-like cells, however, is not necessarily limited to these ranges. Rather, a therapeutically effective amount, as used herein, refers to an amount sufficient to accelerate the wound healing process, as described herein. To this end, any number of cells may be administered such that they achieve the effects contemplated herein.

In further embodiments of the present invention, a therapeutically effective amount of a cell lysate of either MSCs or the myofibroblast-like cells of the present invention may be directly administered at or near the wound site of the patient to accelerate wound healing and minimize scar tissue formation. Most preferably, the cell lysate of the myofibroblast-like cells are administered. As provided herein, each of the MSCs and myofibroblast-like cells express and secrete one or more of, at least, IL-8, IL-6, VEGF, SDF-1, CXCL5, MMP1, CXCL6, COL4A4, MMP13, CYP7B1, ADAMDEC1, SLC6A1, CXCL1, PF4V1, CXCL3, CH25H, SFRP2, DARC, HCK, ERC2, CLIC6, BCL8 or combinations thereof. While not intended to be bound by theory, these cytokines, particularly IL-6 and IL-8 are thought to control MSC recruitment and differentiation, while CXCL5 is thought to control the proliferation of pancytokeratin positive cells. Accordingly, a therapeutically effective amount of either MSCs or myofibroblast-like cell lysate, including the associated cytokines thereof, may be prepared and directly administered subcutaneously at or near the wound area of the patient. The therapeutically effective amount refers to an amount sufficient to accelerate the wound healing process, as described herein, and provides for reduced scar tissue formation.

The MSC lysate or myofibroblast-like cell lysate may be isolated using any methods known in the art. In a non-limiting example, the cell lysate may be prepared using 5×10⁶ cells per 30-50 mm² of the wound. These cells may be sonicated and centrifuged into a cell pellet. The pellet is then re-suspended in phosphate buffer saline and the entire lysate is then administered in accordance with the teachings herein. The therapeutically effective amount of MSCs, however, is not necessarily limited to the foregoing. Rather, a therapeutically effective amount, as used herein, refers to an amount sufficient to accelerate the wound healing process, as described herein. To this end, any number of cells may be lysated and administered such that they achieve the effects contemplated herein.

In even further embodiments of the present invention, a therapeutically effective amount of a KCM containing composition may be directly administered at or near the wound site of the patient to accelerate wound healing and minimize scar tissue formation. In further embodiments the KCM composition may be co-administered with one or more MSC or myofibroblast-like cells of the present invention.

In further alternative embodiments, a therapeutically effective amount of one or more of the cytokines associated with KCM and/or myofibroblast-like cells of the present invention may be directly administered at or near the wound site of the patient to achieve the objectives herein. These cytokines may, optionally, be administered with the stem cells of the present invention. Such cytokines may include, but are not limited to, one or more of IL-8, IL-6, VEGF, SDF-1, CXCL5, MMP1, CXCL6, COL4A4, MMP13, CYP7B1, ADAMDEC1, SLC6A1, CXCL1, PF4V1, CXCL3, CH25H, SFRP2, DARC, HCK, ERC2, CLIC6, BCL8 or combinations thereof. In further embodiments the cytokines may be co-administered with one or more MSC or myofibroblast-like cells of the present invention.

In each of the foregoing embodiments, a therapeutically effective amount of the cells and compositions may be formulated for subcutaneous administration at or near the wound site. Such a subcutaneous administration may be provided by a suspension of the cells or lysate of the present invention wherein the suspension is injected underneath the skin of the patient at or near the wound site. The present invention, however, is not limited to this method of administration and any method of administering cells or compositions of the present invention is applicable. The compositions of the present invention may, therefore, be formulated with any pharmaceutically acceptable carrier or diluent. In one embodiment, the pharmaceutically acceptable carrier or diluent is liquid or semi-solid. In alternative embodiments, for example, non-synthetic matrix proteins like collagen, glycosaminoglycans, and hyaluronic acid, which are enzymatically digested in the body, are useful for delivery (see U.S. Pat. Nos. 4,394,320; 4,472,840; 5,366,509; 5,606,019; 5,645,591; and 5,683,459) and are suitable for use with the present invention. Other implantable media and devices can be used for delivery of the cells of the invention in vivo. These include, but are not limited to, sponges; fibrin gels; scaffolds formed from sintered microspheres of polylactic acid (PLA), polylglycolic acid polymers (PGA), polycaprolactic acid polymer (PCA), co-polymers thereof; nanofibers formed from native collagen; as well as other proteins or matrices known in the art to deliver cell types and biological agents. One of ordinary skill in the art would appreciate that there are other biocompatible/biodegradable carriers useful for delivering the cells of the present invention.

The compositions of the present invention may be delivered by several means, including, without limitation, an injection into the desired part of the subject's body (e.g., subcutaneously), surgical placement, or delivery by a syringe, catheter, trocar, cannulae, stent (which can be seeded with the cells), etc.

In further alternatives, the cells and compositions of the present invention may be topically or subcutaneously applied and covered with a bandage or dressing. Alternatively, the cells of the present invention may be applied directly to the dressing or bandage and the bandage/dressing placed such that the cells contact and are provided to the wound. To this end, the present invention is not limited as to the method of administering the cells to the wound site. Rather, any method known in the art or understood by one of ordinary skill in the art may be employed.

In alternative embodiments of the present invention, the cells of the present invention may be co-administered with one or more biologically active agents. These biologically active agents can include, without limitation, medicaments, growth factors, vitamins, mineral supplements, substances used for the treatment, prevention, diagnosis, cure or mitigation of disease or illness, substances which affect the structure or function of the body, or drugs. The biologically active agents can be used, for example, to facilitate implantation of the composite or cell suspension into a subject to promote subsequent integration and healing processes. To this end, the biologically active agents include, but are not limited to, antifungal agents, antibacterial agents, anti-viral agents, anti-parasitic agents, growth factors, steroids, pain medications (e.g. aspirin, and NSAID, and/or local anesthetic), analgesics, adjuvants, anti-inflammatory agents, angiogenic factors, anesthetics, mucopoly-saccharides, metals, cells, agents useful in the repair of tissue, bone, and vascular injury, and other known wound healing agents. Biologically active agents may also include genes of interest, which can be introduced into or administered with cells of the invention as a gene therapy model. To this end, incorporating herein are the methods of expressing a gene of interest in the stem cells of the present invention or administering a gene of interest such that it is expressed in the somatic cells of the subject.

Suitable antibiotics include, without limitation nitroim-idazole antibiotics, tetracyclines, penicillins, cephalosporins, carbopenems, aminoglycosides, macrolide antibiotics, lincosamide antibiotics, 4-quinolones, rifamycins and nitrofurantoin. Suitable specific compounds include, without limitation, ampi-cillin, amoxicillin, benzylpenicillin, phenoxymethylpenicillin, bacampicillin, pivampicillin, carbenicillin, cloxacillin, cycla-cillin, dicloxacillin, methicillin, oxacillin, piperacillin, ticarcillin, flucloxacillin, cefuroxime, cefetamet, cefetrame, cefixine, cefoxitin, ceftazidime, ceftizoxime, latamoxef, cefo-perazone, ceftriaxone, cefsulodin, cefotaxime, cephalexin, cefaclor, cefadroxil, cefalothin, cefazolin, cefpodoxime, ceftibuten, aztreonam, tigemonam, erythromycin, dirithromycin, roxithromycin, azithromycin, clarithromycin, clindamycin, paldi-mycin, lincomycirl, vancomycin, spectinomycin, tobramycin, paromomycin, metronidazole, tinidazole, ornidazole, amifloxacin, cinoxacin, ciprofloxacin, difloxacin, enoxacin, fleroxacin, norfloxacin, ofloxacin, temafloxacin, doxycycline, minocycline, tetracycline, chlortetracycline, oxytetracycline, methacycline, rolitetracyclin, nitrofurantoin, nalidixic acid, gentamicin, rifampicin, amikacin, netilmicin, imipenem, cilastatin, chloramphenicol, furazolidone, nifuroxazide, sulfadiazin, sulfametox-azol, bismuth subsalicylate, colloidal bismuth subcitrate, gramicidin, mecillinam, cloxiquine, chlorhexidine, dichloro-benzylalcohol, methyl-2-pentylphenol or any combination thereof.

Growth factors that can be incorporated into the composite of the present invention include, but are not limited to, interleukin-8 (IL-8), interleukin-6 (IL-6), vascular endo-thelial growth factor (VEGF), stromal cell-derived factor-1 (SDF-1), chemokine (C—X—C motif) ligand 5 (CXCL5), bone growth factors (e.g., BMP, OP-I), basic fibroblast growth factor (bFGF), acidic fibroblast growth factor (aFGF), nerve growth factor (NGF), epidermal growth factor (EGF), insulin-like growth factors 1 and 2 (IGF-I and IGF-2), platelet-derived growth factor (PDGF), tumor angiogenesis factor (TAF), corticotropin releasing factor (CRF), transforming growth factors alpha and beta (TGF-α and TGF-β), granulocyte-macrophage colony stimulating factor (GM-CSF), the interleukins, and the interferons.

Suitable anti-inflammatory compounds include the compounds of both steroidal and non-steroidal structures. Suitable non-limiting examples of steroidal anti-inflammatory compounds are corticosteroids such as hydrocortisone, cortisol, hydroxyltriamcinolone, alpha-methyl dexamethasone, dexametha-sone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluocinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, difluorosone diacetate, fluocinolone, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclo-pentylpropionate, hydrocortamate, meprednisone, paramethasone, prednisolone, prednisone, beclomethasone dipropionate, triamcinolone. Mixtures of the above steroidal anti-inflammatory compounds can also be used.

Non-limiting examples of non-steroidal anti-inflammatory compounds include nabumetone, celecoxib, etodolac, nimesulide, apasone, gold, oxicams, such as piroxicam, isoxicam, meloxicam, tenoxicam, sudoxicam, and CP-14,304; the salicylates, such as aspirin, disalcid, benorylate, trilisate, safapryn, solprin, diflunisal, and fendosal; the acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; the fenamates, such as mefenamic, meclofenamic, flufenamic, niflumic, and tolfenamic acids; the propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; and the pyrazoles, such as phenylbutazone, oxyphenbutazone, feprazone, azapropazone, and trimethazone.

The present invention is not limited to the foregoing biological agents and methods of administration. Additional agents and methods known by one of ordinary skill in the art may be readily substituted to achieve the same or similar effects and advantages, as provided below.

The cells, compositions, and methods of the present invention are advantageous in that administration of any one or more of these embodiments results in an accelerated healing rate of the wound, relative to typical healing times or healing times without the administration of cells and compositions of the present invention. In one embodiment, the healing times may be between 6-15 days, depending on the size of the wound. In further embodiments, administration of the cells and compositions of the present invention accelerates wound healing by at least 15% relative to the healing rate without the administration of cells of the present invention. In other embodiments, administration of the cells and compositions of the present invention accelerate wound healing by least 40%, relative to the healing rate without the administration of cells and compositions of the present invention.

While not intended to be bound by theory, it is postulated that the hMSCs administered in vivo, as well as those cultured in vitro in the presence of KCM and/or select cytokines, are differentiated into myofibroblast-like cells by communication molecules secreted by keratinocytes. The myofibroblast-like cells then synthesize and secrete growth factors, which in turn stimulate keratinocyte proliferation in a reciprocal manner. It is further postulated that these cytokines, particularly IL-6 and IL-8 control MSC recruitment and differentiation into myofibroblast-like cells. Furthermore, observed increases in CXCL5 (also known as ENA78) in the differentiated stem cells, which is a known stimulator of keratinocytes, may explain the large increase in pancytokeratin positive cells observed upon immunohistochemical analyses of wound areas from animals receiving such cells. By administering hMSCs and/or myofibroblast-like cells at wound site, the present invention ensures that an adequate number of myofibroblasts were available and contributed to proper wound closure. Additionally, administration of cell lysates, particularly those of hMSCs and myofibroblast-like cells, provide for optimal proliferation of both myofibroblast cells and pancytokeratin positive cells within the wound.

The high contractile force generated by myofibroblasts is beneficial for physiological tissue remodeling and minimizes scar tissue formation. Myofibroblasts produce and modify the extracellular matrix (ECM), secrete angiogenic and pro-inflammatory factors, and stimulate epithelial cell proliferation and invasion. In accordance with the foregoing, the stem cells or MSCs of the present invention undergo myofibroblast-like differentiation either in vivo or in vitro and stimulate local keratinocytes and fibroblasts. Granulation tissue fibroblasts (myofibroblasts) develop several ultra-structural and biochemical features of smooth muscle (SM) cells, including the presence of microfilament bundles and the expression of alpha-SM actin, the actin isoform typical of contractile vascular SM cells. Thus, the stem cells and MSCs of the present invention participate in several important areas of wound repair generation of myofibroblasts, formation of a matrix of appropriate tensile strength upon which new layers of dermis and epidermis are formed and supporting neovascular structures in the repaired wound. As such, the stem cells, MSCs and lysates of the present invention are useful for regenerative purposes particularly for healing of both acute and chronic wounds with minimal scar tissue formation.

### EXAMPLES

### Example 1

### Materials and Methods

Human bone marrow was obtained commercially (Cambrex, Walkersville, Md.) and processed in the lab to isolate human mesenchymal stem cells using MesenCult basal media for MSCs with hMSC stimulatory suppliments and FBS for hMSCs (StemCell Technologies Inc. Vancouver, BC) and later expanded in minimal essential alpha medium (Invitrogen). The multipotency of isolated mesenchymal stem cells was confirmed by differentiating them into adipocytes (adipogenic induction medium containing insulin, dexamethasone and indomethacin), osteocytes (osteogenic induction medium containing dexamethasone, beta glycerophosphate, L-ascorbic acid) and myocytes (treated with 5-azacytidine for 24 hrs and cultured for 21 days). The mice (strain: nu/nu, gender: females, age: 4-5 weeks from Taconic farms, NY) were anesthetized and the skin surface was sterilized with alcohol wipes.

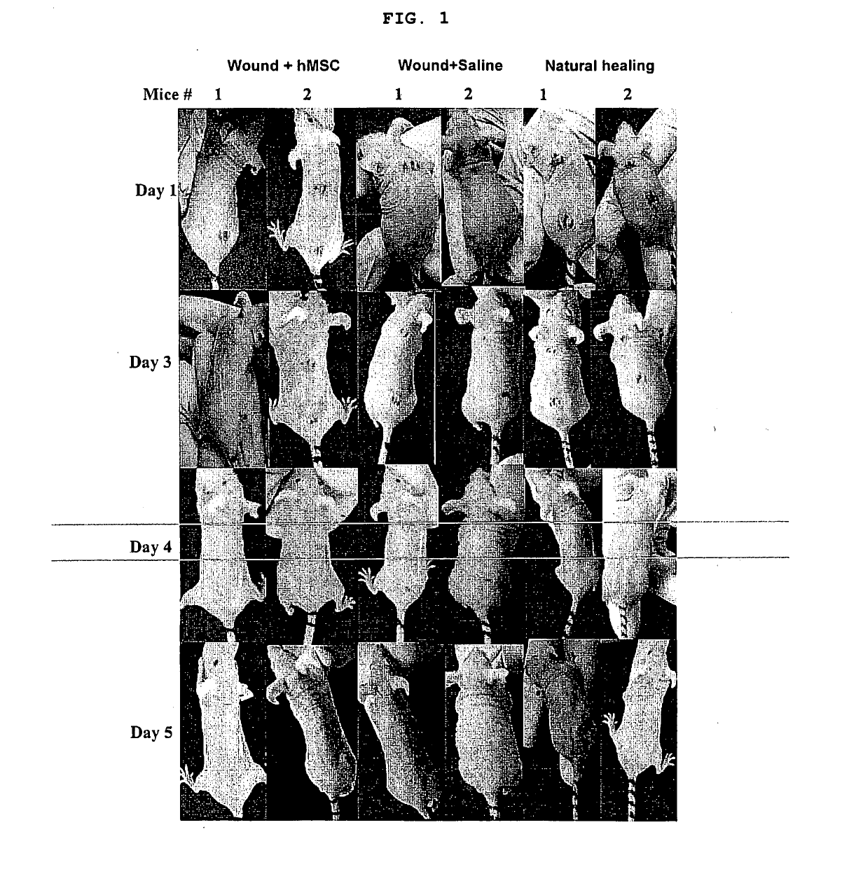

Two wounds (approximate area 0.7 cm) were made in the back of each mouse using a sterile needle (FIG. 1). The wounds were sterilized using alcohol wipes. 5×10⁵ human mesenchymal stem cells were injected subcutaneously near each wound (1×10⁶ cells/mice) in experimental group. Saline (100 uL) was injected subcutaneously near the wounds in the control group.

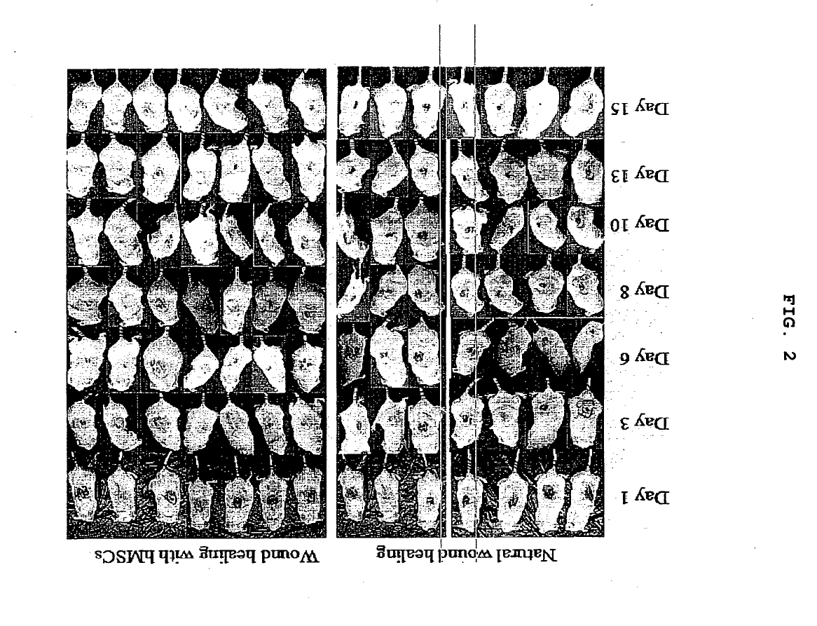

Excision wounds were also created in a diabetic NOD/SCID mice model (n=8) in accordance with the foregoing (FIG. 2). Natural wound healing with saline injected at the wound site served as the control (n=8).

The effectiveness of hMSC cell lysate in the wound healing in diabetic and in normal mice was also evaluated. The hMSC cell lysate prepared using 5×10⁶ cells (per mice). Cell lysate was prepared by sonicating the cell pellet, resuspended in phosphate buffer saline, six bursts for 30 seconds at half max setting. The resultant lysate was injected near excision wound in the normal (n=5) and diabetic mice (n=8).

### Results

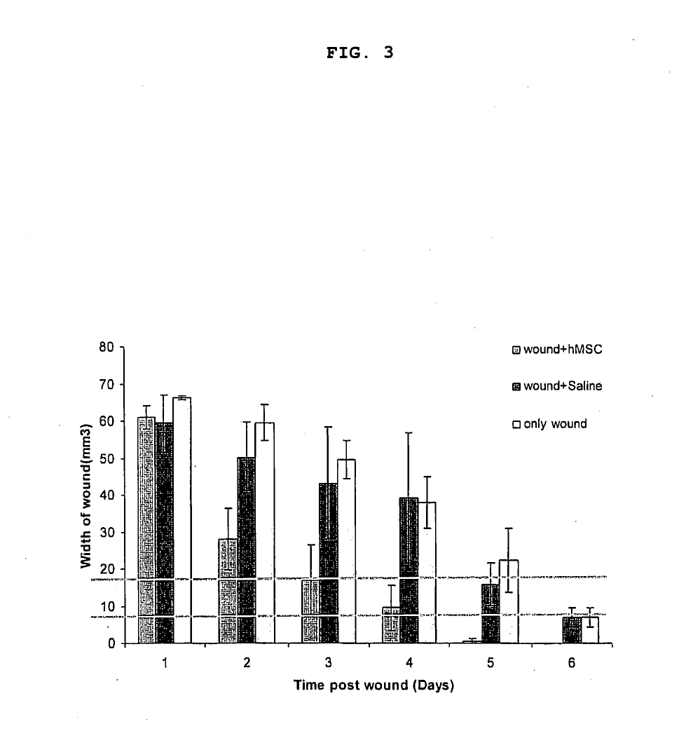

Wounds in 1-MSC-injected group rapidly healed within two days with little or no visible scar (FIGS. 1-3). The control mice injected with saline took seven days for wound healing and there was a visible scar. The skin around the healed wound was surgically removed from anesthetized mice and analyzed by histopathological analysis. Re-epithelialisation and restoration of the normal skin morphology was observed in the mice injected with hMSCs as compared to the natural healing in control mice as determined by the immunohistochemical analysis.

Our data (FIGS. 1-3) demonstrated that injection of hMSCs around the wound significantly enhanced wound healing in diabetic mice (wound closure observed on day 6) as compared to the natural healing in the diabetic mice (wound closure was observed on day 15). The HMSC injected wounds exhibited rapid wound healing and increased re-epithelialization. Histopathological analysis of the wounds revealed expression of keratin (keratinocyte specific protein) and formed glandular structures suggesting a direct contribution of hMSCs to skin regeneration and repair.

Of interest the hMSC cell lysate significantly enhanced the wound healing as compared to the natural healing in normal and diabetic mice. However, live hMSC treatment was more effective.

Since hMSC cell lysate alone initiates the rapid wound healing in preclinical animal models (normal and diabetic) this suggests that the cell products obtained from hMSCs could also be potentially used in the treatment of normal and diabetic wounds.

In conclusion this data demonstrates in preclinical models, the effectiveness of human mesenchymal stem cells and the cell products in the treatment of chronic wounds including under disease conditions such as diabetes, where wound healing is severely impaired.

### Example 2

### Materials and Methods

Isolation of BMD-hMSCs and culture conditions—Unprocessed bone marrow (36×10⁶ cells/ml) was purchased from Lonza (Walkersville, Md.). A Ficoll gradient was used for isolation of hMSCs and to eliminate unwanted cell types from bone marrow. Cells were then plated in T75 cm² tissue culture flasks with Mesencult media containing hMSC stimulatory supplements and fetal bovine serum (FBS) for hMSCs. Once cultures were established, several clones were isolated and expanded in culture in the same medium. Established cultures were grown in minimum essential media (α-MEM) containing 10% FBS and penicillin/streptomycin. The cultures were incubated at 37° C. in a humidified atmosphere containing 5% CO₂. Cells were subcultured every 4 to 5 days and aliquots from passage 2 to 8 were frozen in liquid nitrogen for use. Cell surface markers expressed on these cells were determined by flow cytometry using FITC labeled Abs (BD Biosciences, San Jose, Calif.) and include Strol, CD105, CD90, HLA-ABC and CD44 while they were negative for CD45, HLA-DR and CD11b.

Multi lineage differentiation—Expanded cultures of hMSCs were analyzed for myogenic, osteogenic and adipogenic differentiation in vitro to determine multipotency according to standard conditions.

In-vitro Migration assay—Migration assays were carried out. Briefly, Falcon tissue culture plates with 24 wells along with a companion Falcon cell culture inserts were used for the migration assay. CM from keratinocytes (collected after overnight culture in fresh growth medium) or keratinocytes (1×10⁴) were plated in the bottom chamber and incubated overnight at 37° C., and 5% CO₂. Next day, the insert was placed aseptically in the well with flanges resting in the notches on the top edge of each well. Naive hMSCs (2×10⁴) were plated on the top. The assay was terminated and hMSCs that had migrated through the membrane (8 μm pore size) were then stained (FIG. 1B) (after removal of cells remaining on top with a wet Q-tip) using crystal violet prepared with methanol and formaldehyde.

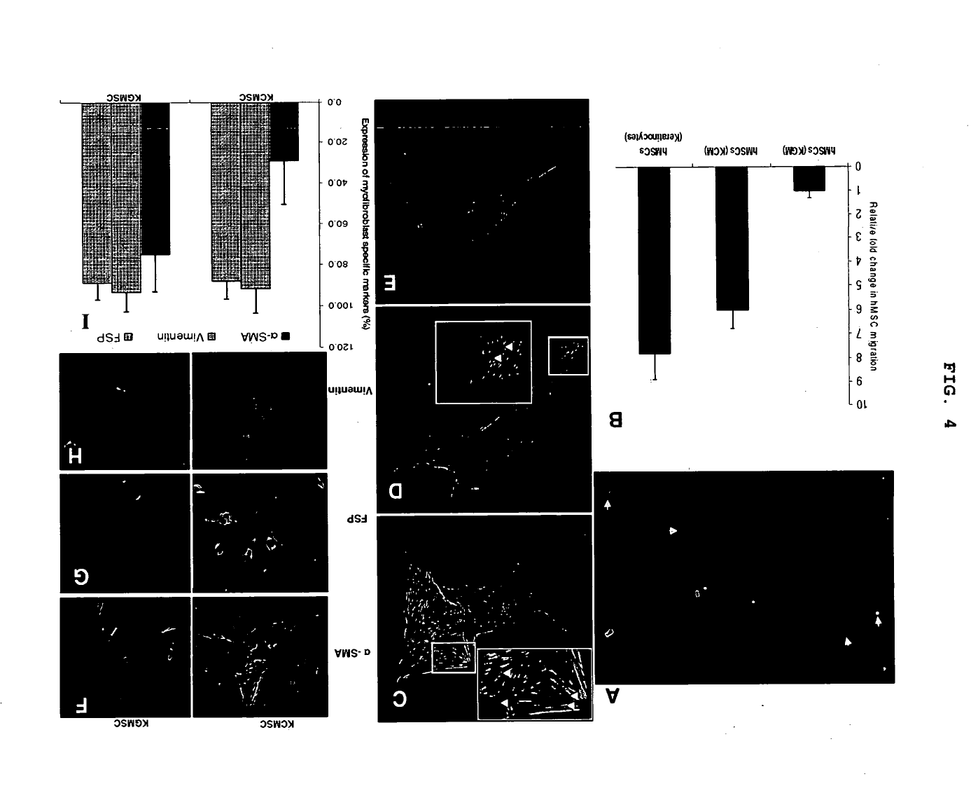

In-vivo Migration assay—Fluorescent dye (CFDASE) labeled 5×10⁵ hMSCs were injected at the periphery of wounded skin subcutaneously. Saline (100 μL) was injected subcutaneously near the wounds as a control. After 48 hr wound areas were excised and immediately fixed and embedded in paraffin wax. Thin sections were cut and placed onto glass slides for staining with DAPI and observed under fluorescence microscope (FIG. 4A).

Exposure of hMSCs to Keratinocyte Conditioned Medium (KCM)—Normal human epithelial primary keratinocyte cell line (NHEK; C-12001) derived from foreskin (−500,000 cells) was obtained from Promocell GmbH (Germany) and cultured in Keratinocyte Growth medium (KGM; C-20011, Promo cell GmbH, Germany). Conditioned medium (CM) from these human keratinocytes was harvested following overnight culture, centrifuged at 3000 rpm for 5 min and supernatant passed through Millipore sterile 50 ml filtration system with 0.45 μm PVDF membrane. hMSCs were exposed to fresh keratinocyte conditioned media (KCM) repeatedly for 30 days with the KCM being changed every third day.

Conditioned medium concentrate—hMSCs were exposed for 30 days to KCM to generate KCMSCs and to KGM to generate KGMSCs and conditioned medium from KCMSC and KGMSC was collected and further concentrated (50 times) by Amicon Ultra centrifugal Filter unit with ˜5 kDa cut-off (Amicon Ultra-15, UFC903008; Millipore, Mass.) following manufacturer's instructions. Concentrate (100 μl) From KCMSC conditioned medium concentrate {KCMSC(CMC)}, KGMSC conditioned medium concentrate {KGMSC(CMC)}, MSC conditioned medium concentrate {MSC(CMC)}, was injected in the periphery of each wound.

Cell Lysate Preparation: Cultured early passage cells (hMSC, WI38) were trypsinized and pellet was collected to prepare Cell lysate. Cells were sonicated for 30 sec (6 times), while maintaining it on the ice. Protein concentration in the lysate was detected by using standard Bradford method. Cell lysate was injected in the wound periphery subcutaneously.

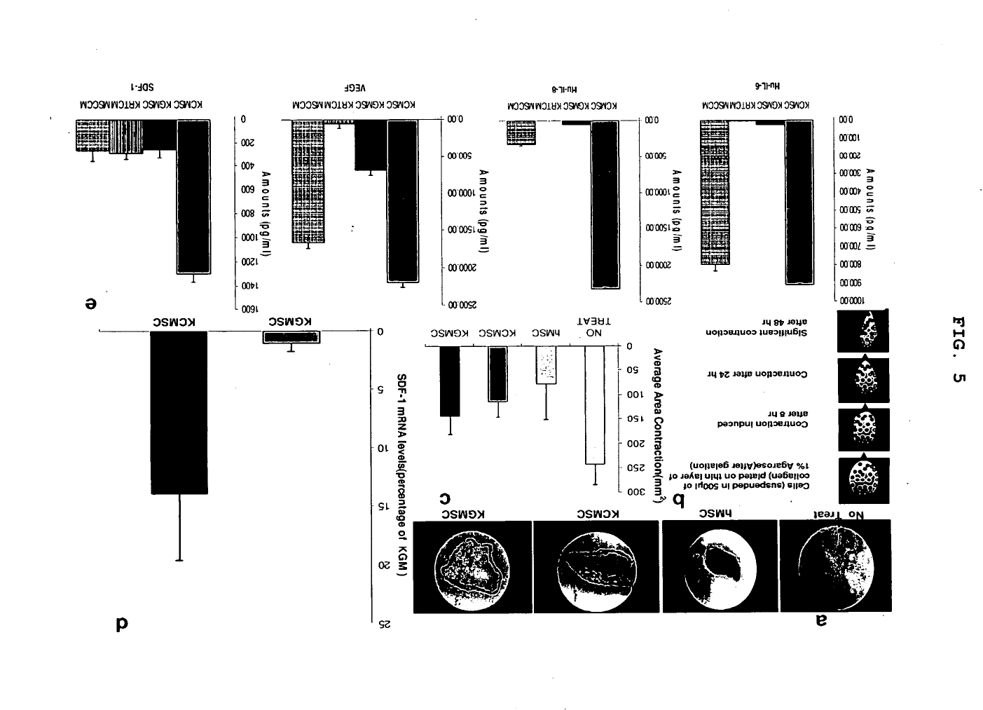

Floating collagen gel contraction (FCGC) assay: FCGC assay was performed. Briefly, one volume of a rat tail collagen (BD Biosciences, Bedford, Mass.) stock solution was brought to physiological ionic strength with one-ninth volume of NaHCO₃. DMEM with FBS was added to the salt-balanced collagen stock to yield a solution of 0.555 mg/ml collagen with 10% FBS, pH 7.4. The collagen solution was maintained on ice. Meanwhile, wells of 24 well tissue culture plates were coated with 1% agarose and allowed to solidify. 6×10⁴ cells (hMSC, KCMSC and KGMSC) were mixed in rat tail collagen (50 μl/well) in a volume ratio of 1:9 to yield gels with a final concentration of 0.5 mg/ml of collagen and added to each well, polymerized in the tissue culture incubator, and induced to float by addition of Dulbecco's Modified Eagle's Medium (DMEM; Invitrogen) with 10% FBS. After 2 h, floatation of gel was confirmed visually and the gels returned to the tissue culture incubator to initiate contraction for 24-48 h (FIG. 5a-c). Symmetry of contracted gel was compared between No treatment, hMSC, KCMSC and KGMSC and measured using the publicly available NIH Image program (U.S. National Institutes of Health, http://rsb.itffo.nih.gov/nih-image/) with an edge enhancement filter.

Skin RNA Extraction—Skin sections within close proximity of the wounded area were peeled out after euthanasia from mice injected with hMSCs or lysates prepared from hMSCs along with naturally healing and normal mice. The resected section was immediately dipped in liquid nitrogen and transferred to a ceramic mortar filled with liquid nitrogen where skin sections were ground with a cold pestle until it turned into amorphous powder. The powder was scraped in to a pre-chilled falcon tube in a dry ice containing TRIzol (Invitrogen; Carlsbad Calif. USA) reagent (1 ml/40-100 mg of tissue weight). The tube was vortexed vigorously and transferred into a pre-cleaned homogenizer and homogenized with 20 up and 20 down strokes. The homogenized solution was incubated for 5 min at room temperature followed by addition of molecular biology grade chloroform (Sigma, 40 μl/1.5 ml of TRIzol Reagent) and mixed. The solution was incubated for an additional 5 min at room temperature and centrifuged (eppendorf table top centrifuge) at 12000×g (15-17 min at 4° C.). The upper aqueous phase was collected in a new sterile falcon tube and isopropyl alcohol was added (1:1), mixed thoroughly and incubated (10 min at RT) followed by centrifugation (12,000×g for 10-15 min at 4° C.). Carefully without disturbing the pellet the supernatant was aspirated. The RNA pellet was washed with 50 μl of 70% Ethanol (Prepared in RNase free water (GIBCO, Invitrogen)) and centrifuged (7000×g for 5 min at 4° C.). The RNA pellet was air dried (20 min) and then resuspended in 40 μl (depends on the size of pellet) RNase free water which was stored at −80° C. until used.

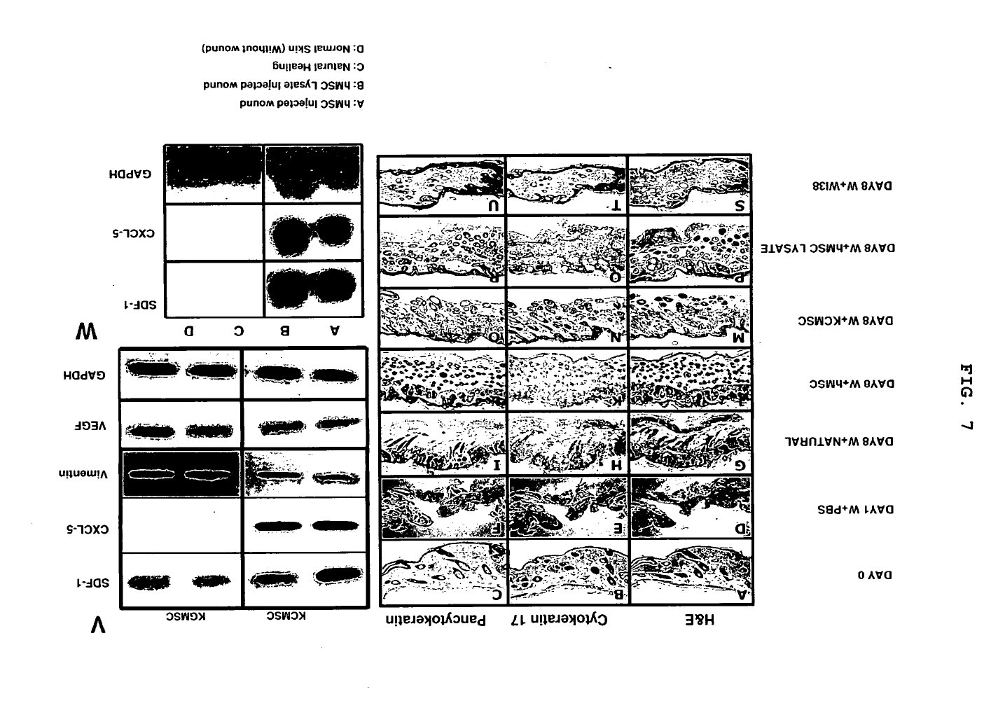

RT-PCR analysis—Total RNA was extracted (RNeasy mini kit from Qiagen (Qiagen Sciences, Md.) from KCMSC and KGMSC. mRNA expression levels of SDF-1, Vimentin and VEGF in KCMSC and KGMSC were determined using quantitative and or semiquantitative RT-PCR analysis using SDF-1,CXCL5, Vimentin and VEGF specific primer (human) sets (Table-1). As an internal control, levels of glyceraldehyde-3-phosphate dehydrogenase (GAPDH) mRNA were quantified from the same RNA sample. Similarly to determine mRNA expression levels of SDF-1 and CXCL5 in hMSC and hMSC lysate injected skin (wounded), naturally healing wounded skin and normal skin, RT-PCR analysis was carried out using SDF-1,CXCL5 and GAPDH (internal control) specific primer (mouse) sets (Table-1). For the reaction, superscript one step RT-PCR (Invitrogen, Carlsbad, Calif.) kit was used. PCR conditions were 94° C. for 15 seconds, 50° C. for 30 seconds, 72° C. for 1 minute, and 30 cycles for each target. The final elongation step was carried out at 72° C. for 7 min. The PCR product was subjected to agarose gel analysis and photographed (FIG. 7V-W) using a Geldoc imager (Bio-Rad XRS).

TABLE I

| | Forward | Reverse |
|---|---|---|
| Primers | | |
| (human) | | |
| SDF-1 | 5′-AGAGATGAAAGGGCAAAGAC-3′ | 5′-CGTATGCTATAAATGCAGGG-3′ |
| | (SEQ ID NO. 1) | (SEQ ID NO. 2) |
| CXCL5 | 5′-TGTTGAGAGAGCTGCGTTGC-3′ | 5′-GTTTTCCTTGTTTCCACCGTCC-3′ |
| | (SEQ ID NO. 3) | (SEQ ID NO. 4) |
| Vimentin | 5′-TGGCACGTCTTGACCTTGAA-3′ | 5′-GGTCATCGTGATGCTGAGAA-3′ |
| | (SEQ ID NO. 5) | (SEQ ID NO. 6) |
| VEGF | 5′-GAAGTGGTGAAGTTCATGGATGTC-3′ | 5′-CGATCGTTCTGTATCAGTCTTTCC-3′ |
| | (SEQ ID NO. 7) | (SEQ ID NO. 8) |
| GAPDH | 5′-TCCACCCATGGCAAATTCC-3′ | 5′-AGCATCGCCCCACTTGATT-3′ |
| | (SEQ ID NO. 9) | (SEQ ID NO. 10) |
| Primers | | |
| (Mouse) | | |
| SDF-1 | 5′-GAGAGCCACATCGCCAGA G-3′ | 5′-TTTCGGGTCAATGCACACTTG-3′ |
| | (SEQ ID NO. 11) | (SEQ ID NO. 12) |
| CXCL5 | 5′-TTCATGAGAAGGCAATGCTG-3′ | 5′-CCCAGGCTCAGACGTAAGAA-3′ |
| | (SEQ ID NO. 13) | (SEQ ID NO. 14) |
| GAPDH | 5′-ACCACAGTCCATGCCATCAC-3′ | 5′-TCCACCACCCTGTTGCTGTA-3′ |
| | (SEQ ID NO. 15) | (SEQ ID NO. 16) |

Microarray analysis—Cells were harvested following exposure to KCM or KGM and RNA was isolated using RNeasy mini kit (Qiagen Sciences, Md.). 5 μg of total RNA was processed for micro array analysis following verification of quality at DNA micro array core facility of CINJ/RWJMS. Briefly, the RNA was reverse transcribed and hybridized to Affymetrix Gene Chip® Human Genome U133 Plus 2.0Array Comprised of more than 54,000 probe sets and 1,300,000 distinct oligonucleotide features and analyzes the expression level of over 47,000 transcripts and variants, including 38,500 well-characterized human genes. Comparative analyses of expressed genes that were either down regulated or up regulated under various experimental conditions by greater than 1.5 fold (p<0.05 for upregulated genes, all values expressed in log 2) was carried out using proprietary software Gene Sifter (www.genesifter.net from VizX Labs, Seattle, Wash.). Three independent sets for each of the experimental conditions was carried out and analyzed to control for intra sample variation. Data normalization was performed by applying the RMA method implemented in the library affy of the Bio-conductor software (www.bioconductor.org). Comparative analyses of expressed genes that were either down regulated or up regulated under various experimental conditions by greater than 1.5 fold (permutation p value <0.05 and false discovery rate <0.25 for signal-to-noise ratio (SNR), all values expressed in log 2) was carried out using the GenePattern software available at the Broad Institute.

Pathway analysis was performed by applying the Gene Set Enrichment Analysis software (www.broad.mit.edu/gsea/) and KEGG, a publicly available gene expression analysis software.

Induction of wounds and measurement of wound areas: Mice (strain: male nu/nu, and NODSCID mice; age: 4-5 weeks from Taconic Farms, N.Y.) were anesthetized with ketamine/xylazine and the skin surface was sterilized with alcohol wipes. The NODSCID mice were shaved to expose skin for wounding. Wounds (approximate area 30 to 50 mm²) were made in the back of each mouse. The wounds were covered with transparent adhesive bandage for 48 h post wounding. 5×10⁵ human mesenchymal stem cells were injected subcutaneously in the periphery of each wound in experimental group. Saline (100 μL) was injected subcutaneously near the wounds in one control group and WI38 fibroblast cells were injected in another control group.

Measurement of wound healing was carried out using area of ellipse formula (0.5×length of Major axis) (0.5×length of Minor axis) (π). Wound bearing animals were housed individually during the course of the experiment.

Immunofluorescence analysis—The following antibodies were used for immunofluorescence studies: monoclonal Anti Vinculin antibody (1:200, P1951; Sigma-Aldrich); α-Smooth Muscle Actin (1:250; mouse monoclonal clone 1A4, A2547); Fibroblast Surface Protein (1:250; mouse monoclonal clone 1B10, F4771); Vimentin (1:200, clone VIM-13.2, V5255; Sigma-Aldrich). Primary antibodies were visualized with Alexa Fluor488P (Ab′)2, IgG (H+L) (1:400; Molecular Probes) and Alexa Fluor 555 goat anti-mouse IgM (1:400; Invitrogen). Phalloidin-Tetramethylrhodamine β isothiocyanate (50 μg/ml) was obtained from Sigma-Aldrich and 4′,6-diamidino-2-phenylindole (DAPI) from Vector Laboratories.

Immunostaining was performed on cells grown on sterilized coverslips in 12-well plates. The cells were fixed in 4% paraformaldehyde (at room temperature, 10 min), washed with 1×PBS followed by permeabilization with 0.1% Triton X-100 for 10 min. Cells were again washed, exposed to blocking medium (α-MEM) with 10% FBS, and then incubated with primary antibodies (Vinculin™, α-SMA, FSP, vimentin) for 1 h at room temperature. After 5 subsequent washes with PBS for 5 min each, cells were immunostained with secondary antibodies at a dilution of 1:400 in a blocking medium. Secondary antibodies used were Alexa Fluor 488P (Ab′)2, IgG (H+L), and Alexa Fluor 555 anti-mouse IgM (1:400; Sigma-Aldrich). When cells were concomitantly stained for actin stress fibers, they were incubated with Phalloidin-Tetramethylrhodamine B isothiocyanate (50 μg/ml) with the secondary antibody. Following further washes, the cells were counterstained with the nuclear dye TOPRO-3 iodide (1:1,000; Invitrogen, Molecular Probes) in PBS (Life Technologies) at room temperature in the dark, followed by subsequent washing. Cells were embedded in VectaShield mounting medium with DAPI and examined with the fluorescence and confocal microscope. The nïve and differentiated hMSCs were quantitated for expression of myofibroblast specific markers. Total cell number was obtained by counting the total number of DAPI stained nuclei under the microscope. Percentage of marker expressing cells to the total number of the cells was calculated.

Immunohistochemistry—Wound areas were excised and immediately fixed for 24 h before processing through graded series of alcohols and embedded in paraffin wax. Thin sections (4 microns) were cut and placed onto glass slides for staining. Antigen retrieval was performed for over 70 min at pH-8 using EDTA. Antibody staining using 100 μl of antibody at a dilution of approx 1:1000 (anti-) was applied to the slides and incubated at 37° C. for 60 min. Primary antibodies were diluted with Dako-diluent (Dako, Carpinteria, Calif.). Tissue sections were rinsed in buffer. The diluted biotinylated secondary antibody was applied to the tissue sections and incubated for 12 min at 37° C. Hematoxylin was used as a tissue counterstain.

### Results

Prolonged exposure to KCM induces differentiation of BMD-hMSCs with expression of dermal myofibroblast/myofibroblast markers—KCM induced expression of cytoskeletal markers vinculin and F-actin filaments in differentiated hMSCs indicated dermal myofibroblast-like differentiation in KCMSC (FIG. 4C-E). KCMSCs also show punctate vinculin staining, characteristic of focal adhesions. The focal adhesions appear to hold down actin stress fibers, as evidenced by the merge of the vinculin and phalloidin staining for F-actin (FIG. 4C). Further in the study to validate phalloidin staining of the visible stress fibers was actually due to the presence of the actin filaments an antibody to alpha smooth actin was used. As a result KGMSCs expressed little alpha smooth muscle actin, while KCMSC expressed increased amounts of these markers. (FIG. 4F). Quantitative analysis revealed that on average 75.3% of KCMSCs expressed alpha smooth actin where as only 29.2% of the KGMSCs expressed this marker (FIG. 4I). Also myfibroblast markers such as Vimentin and fibroblast surface protein expression was observed in KCMSC and KGMSC (FIG. 4G-H). The induction of α-SMA, FSP, F-actin and punctate Vinculin staining is all consistent with KCM inducing the differentiation of hMSCs into dermal myofibroblast-like cells (FIG. 4C-H).

Migration of hMSCs towards keratinocytes—hMSCs were assayed for their ability to migrate toward keratinocytes or KCM in a transwell chamber migration assay. The hMSCs were found to migrate toward keratinocytes as well as to KCM in greater numbers than to control medium (FIG. 4B). Thus, exposure to secreted factors such as cytokines present in KCM may “prime” hMSCs to respond and migrate towards keratinocytes.

Gene expression analysis of KCMSC: The effect of prolonged exposure to KCM vs control media (KGM) on hMSCs on gene expression was assayed by gene expression profiing using Affymetrix U133 Plus 2 arrays. Amongst the genes most upregulated by exposure to KCM were genes associated with cytokine signaling (CXCL5, CXCL12 (SDF-1), IL-6, IL-8, etc), cell adhesion, and Myofibroblast differentiation. These data demonstrated KCM exposure induces production of a set of cytokines known to be important in wound healing, and a set of genes associated with myofibroblast differentiation. Pathway analysis using GSEA and KEGG, confirmed that the following pathways were increased by greater than 20 fold in KCMSC versus KGMSC: cytokine-cytokine receptor interactions, cell adhesion molecules, tight junctions, NF-kB target genes, chemokine activity and extra cellular regions.

Cytokine profile of KCMSC—Multiplex assay was performed to determine cytokine profile of conditioned medium from keratinocytes, MSCs and from KCMSCs. Keratinocytes secreted high levels of cytokines including IL-6, 8, etc. that have been previously shown to attract human MSCs. Augmentation of cytokine secretion was seen upon culturing MSCs in KCM. Greatest increase in secreted levels were observed for IL-6, IL-8, SDF-1 and VEGF among the panel of 12 cytokines examined in conditioned medium collected from KCMSC versus KGMSC (FIG. 5e). These data are consistent with the gene-expression microarray data showing increased expression of these cytokines in KCMSCs. RT-PCR analysis was also performed to independently verify increased production of SDF-1 mRNA (FIG. 5d).

Wound healing—To evaluate effect of human bone marrow derived MSCs on wound healing, wound area following administration of 5×10⁵ MSCs, 5×10⁵ WI-38 cells, or saline control was measured over 15 days in the nude mouse model and 25 days in the NOD/SCID model. Quantification of the wound area indicated that mice administered MSCs showed accelerated wound healing in both models, compared with either WI-38 treated or saline-treated controls. In the nude mouse model, healing was completed between 6-8 days, while in the untreated and in the WI38 treated groups, healing required 13-14 days. In the NOD/SCID model, animals treated with MSCs completed wound healing in 11-13 days while other groups took longer than 22 days.

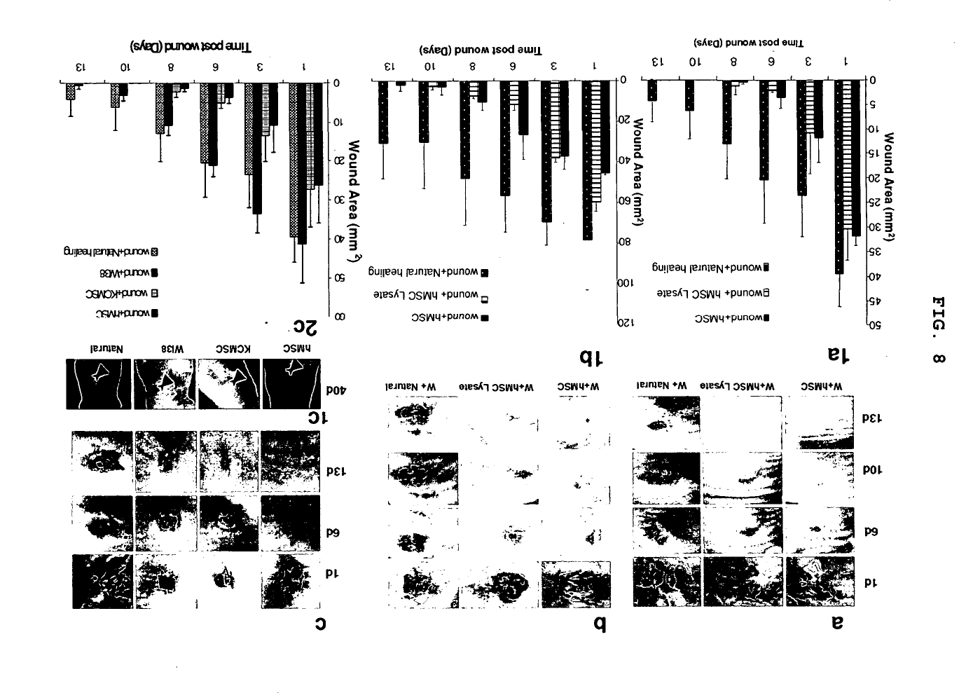

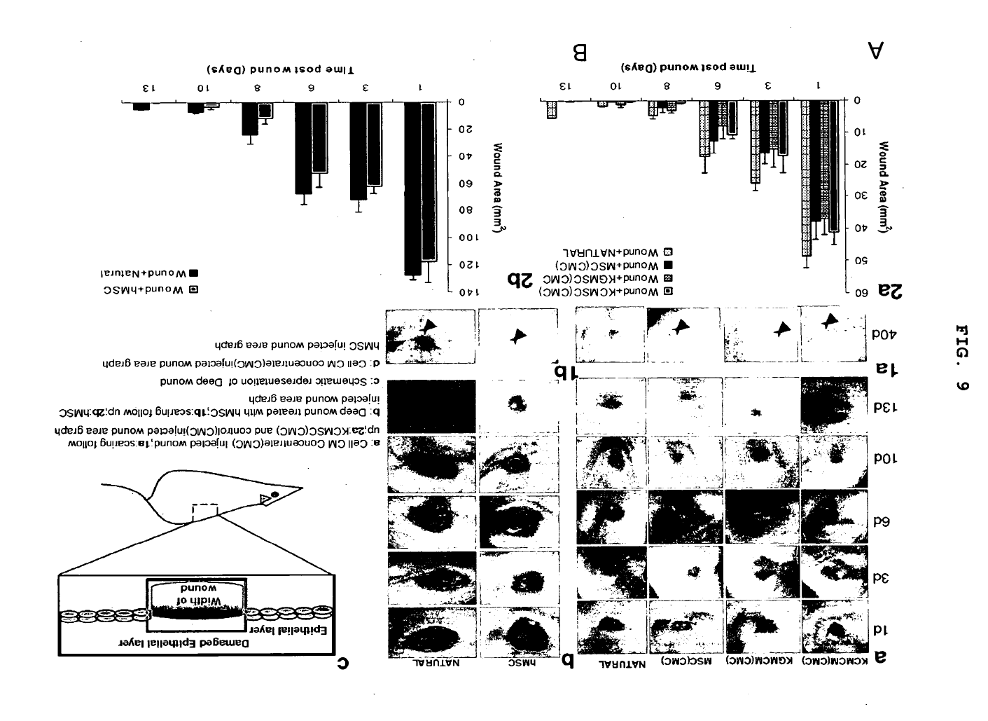

To determine what role secreted factors from hMSCs played in accelerating wound healing in this setting, this assay was performed using concentrated conditioned media from KCMSCs, or whole cell lysates of these cells, compared to KGMSC. Interestingly, lysates prepared from MSCs and concentrated conditioned medium from KCMSCs were also able to accelerate wound healing in the nude mouse model (FIG. 8-FIG. 9).

Immunohistochemical analyses of wound healing—H&E stains and immunohistochemical staining indicated that administration of MSCs near wound sites led to superior regeneration of the skin structure as compared with sections prepared from animals either untreated or treated with control WI38 cells. Figure shows restoration of both dermis and epidermis in skins of mice treated with hMSCs as compared with controls (FIG. 7A-U). Large number of pancytokeratin positive cells were observed in the dermis of hMSC treated wounds indicating that administration of MSCs at wound site may have induced increased proliferation of keratinocytes.

hMSC treated animals have decreased scar formation after wound healing—The long term response to wound healing was monitored for up to 40 days in animals subject to wounding and treated with MSCs, WI-38, lysates and concentrated conditioned medium. In animals treated with MSCs, healing occurred without residual long term scarring while in all other groups, including animals treated with lysates or concentrated conditioned medium from KCMSCs, healing was accompanied by significant residual scarring (FIGS. 8.1c and 9.1a-1b).

What is claimed is:

1. An isolated cell population comprising:
differentiated mesenchymal stem cells having myofibroblast-like characteristics wherein the stem cells are differentiated through exposure to one or more communication molecules from a keratinocyte conditioned medium.

2. The isolated cell population of claim 1 wherein the differentiated mesenchymal stem cells are differentiated human mesenchymal stem cells.

3. The isolated cell population of claim 1 wherein the differentiated mesenchymal stem cells are a differentiated bone marrow derived human mesenchymal stem cells.

4. The isolated cell population of claim 1 wherein the differentiated mesenchymal stem cells are differentiated autologous mesenchymal stem cells.

5. The isolated cell population of claim 1 wherein the mesenchymal stem cells are differentiated through exposure with one or more communication molecules for approximately 10-30 days.

6. The isolated cell population of claim 1 wherein the communication molecules are selected from the group consisting of IL-6, IL-8, VEGF, SDF-1, CXCL5, and combinations thereof.

7. The isolated cell population of claim 1 wherein the differentiated mesenchymal stem cells exhibit at least one cytoskeletal marker associated with a myofibroblast cell type.

8. The isolated cell population of claim 7 wherein the cytoskeletal marker is selected from the group consisting of α-smooth muscle actin, vinculin, vimentin, F-actin, fibroblast surface protein and combinations thereof.

9. The isolated cell population of claim 7 wherein at least 29% of the differentiated mesenchymal stem cells express α-smooth muscle actin.

10. The isolated cell population of claim 7 wherein at about 75% of the differentiated mesenchymal stem cells express α-smooth muscle actin.

11. The isolated cell population of claim 1 wherein the differentiated mesenchymal stem cells express one or more cytokines associated with a myofibroblast cell type.

12. The isolated cell population of claim 11 wherein the cytokines are selected from the group consisting of IL-6, IL-8, SDF-1, CXCL5, VEGF, MMP1, CXCL6, COL4A4, MMP13, CYP7B1, ADAMDEC1, SLC6A1, CXCL1, PF4V1, CXCL3, CH25H, SFRP2, DARC, HCK, ERC2, CLIC6, BCL8 and combinations thereof.

13. The isolated cell population of claim 11 wherein the differentiated mesenchymal stem cells exhibit expression of about 0.0-2,300.0 pg/ml of each of the one or more cytokines.

14. The isolated cell of claim 11 wherein the differentiated mesenchymal stem cells exhibit expression of between 800.0-900.0 pg/ml of IL-6.

15. The isolated cell of claim 11 wherein the differentiated mesenchymal stem cells exhibit expression of between 450.0-2,300.0 pg/ml of IL-8.

16. The isolated cell of claim 11 wherein the differentiated mesenchymal stem cells exhibit expression of between 1,600.0-2,300.0 pg/ml of VEGF.

17. The isolated cell of claim 11 wherein the differentiated mesenchymal stem cells exhibit expression of between 225.0-1,300.0 pg/ml of SDF-1.

18. A method for differentiating a stem cell into a cell exhibiting myofibroblast-like properties comprising:
isolating one or more mesenchymal stem cells within a population of stem cells;
exposing the mesenchymal stems cells to one or more communication molecules from a keratinocyte conditioned medium; and
differentiating the mesenchymal stem cells into a myofibroblast-like cell.

19. A method for healing a wound in a patient comprising:
isolating a population of mesenchymal stem cells;
differentiating the mesenchymal stem cells into myofibroblast-like cells; and
administering a therapeutically effective amount of the differentiated mesenchymal stem cells to a wound site.

20. A method for healing a wound in a patient comprising:
isolating a population of mesenchymal stem cells;
exposing the population of mesenchymal stem cells to one or more communication molecules from a keratinocyte conditioned medium;
differentiating the mesenchymal stem cells into myofibroblast-like cells; and
administering a therapeutically effective amount of the differentiated mesenchymal stem cells to a wound site.

21-42. (canceled)

43. A method for healing a wound in a patient comprising:
isolating a population of mesenchymal stem cells; and
administering a therapeutically effective amount the mesenchymal stem cells to a wound site.

44. A composition for healing a wound in a patient comprising:
a cell lysate prepared from a population myofibroblast-like cells derived from mesenchymal stem cells wherein the mesenchymal stem cells were differentiated into myofibroblast-like cells from exposure to one or more communication molecules from a keratinocyte conditioned medium.

45. A composition for healing a wound in a patient comprising:
a cell lysate prepared from a population mesenchymal stem cells.

46-51. (canceled)

52. A method for healing a wound in a patient comprising:
isolating a population of mesenchymal stem cells;
differentiating the mesenchymal stem cells into myofibroblast-like cells;
isolating a cell lysate of the myofibroblast-like cells; and
administering a therapeutically effective amount of the cell lysate to a wound site.

53. A method for healing a wound in a patient comprising:
isolating a population of mesenchymal stem cells;
isolating a cell lysate of the mesenchymal stem cells; and
administering a therapeutically effective amount of the cell lysate to a wound site.

54-63. (canceled)
